(19) United States
(12) Patent Application Publication
(10) Pub. No.: US 2009/0317408 A1
(43) Pub. Date: Dec. 24, 2009

(54) LIGAND CONJUGATED THERMOTHERAPY SUSCEPTORS AND METHODS FOR PREPARING SAME

(75) Inventors: Robert Ivkov, Chelmsford, MA (US); Cordula Gruttner, Guestrow (DE); Knut Meuller, Kuehlungsborn (DE); Joachim Teller, Mistorf (DE); Fritz Westphal, Behnkenhagen (DE); Allan Foreman, Epping, NH (US); David B. Kanne, Corte Madera, CA (US); Hsiao-Ling Chin, Moraga, CA (US); Paul C. Chinn, Carlsbad, CA (US)

Correspondence Address: PEPPER HAMILTON LLP, ONE MELLON CENTER, 50TH FLOOR, 500 GRANT STREET, PITTSBURGH, PA, 15219, US

(73) Assignee: Aduro BioTech, Berkeley, CA (US)

(21) Appl. No.: 12/335,203

(22) Filed: Dec. 15, 2008

Related U.S. Application Data
(60) Provisional application No. 61/013,412, filed on Dec. 13, 2007.

(51) Int. Cl. A61K 39/395; C07K 16/00; A61P 31/00; A61P 35/00

(52) U.S. Cl. 424/178.1; 530/391.1

(57) ABSTRACT

Magnetic nanoparticles exhibiting enhanced heating ability in thermotherapeutic applications are described, as are several strategies to conjugate such nanoparticles. Methods for using conjugated nanoparticles are also provided.

### CROSS-REFERENCE TO RELATED APPLICATIONS

This application claims priority under 35 U.S.C. §119(e) to U.S. Provisional Patent Application Ser. No. 61/013,412, filed Dec. 13, 2007, the disclosure of which is incorporated by reference in its entirety.

### TECHNICAL FIELD

The present invention relates generally to therapeutic nanoparticle compositions and more specifically to ligand conjugated nanoparticles for use in thermotherapy and methods for preparing such particles.

### BACKGROUND

Conventional treatments for diseases, such as, for example, cancer and some pathogen based diseases, include treatments that are invasive and may be attended by harmful side effects (e.g., toxicity to healthy cells, disruption of normal bodily function) often resulting in a traumatic course of therapy with only modest success. Conventional treatments for cancer, for example, typically include local therapies including surgery followed by systemic therapies such as radiation and/or chemotherapy. These techniques are not always effective, and even if effective, they are characterized by certain deficiencies. For example, surgical procedures can lead to disfigurement and incomplete removal of effected tissue may be associated with a greater risk of cancer recurrence. Radiation therapy and chemotherapy can be physically exhausting to the patient and are not completely effective against recurrence.

Treatment of pathogen-based diseases, as another example, often includes administration of broad-spectrum antibiotics as a first step. This course of action is ineffective against viral pathogens and often eliminates benign intestinal flora in the gut that are necessary for proper digestion of food leading to gastrointestinal distress until the benign bacteria can repopulate. In other instances, antibiotic-resistant bacterial pathogens do not respond to antibiotic treatment. Moreover, therapies designed to treat viral diseases often target only the invading viruses themselves. The cells that the viruses have invaded and commandeered for use in producing additional copies of the virus remain viable. Hence, progression of the disease is merely delayed by the antibiotic treatment, rather than terminated.

An alternative to conventional treatments is immunotherapy, a rapidly expanding approach to treating a variety of human diseases including cancer. The ability to engineer antibodies, antibody fragments, and peptides with altered properties such as antigen binding affinity, molecular architecture, and specificity has expanded their use in therapeutics, as have advances in the chimerization and humanization of murine antibodies to reduce immunogenic responses in humans. In addition, phage display technology, ribosome display, and DNA shuffling have allowed for the discovery of antibody fragments and peptides with high affinity and low immunogenicity for use as targeting ligands. These advances, among others, have made it possible to design immunotherapy regimes with specific antigen binding affinity and specificity with minimal immune response.

Immunotherapeutics fall into at least three classes: (1) deployment of antibodies that target growth receptors, disrupt cytokine pathways or induce complement or antibody-dependent cytotoxicity; (2) directly armed antibodies that include a toxin, a radionuclide, or a cytokine attached to the antibody; and (3) indirectly armed antibodies that are attached immunoliposomes, which contain a toxin or that are attached to an immunological cell effector (bispecific antibodies). The disadvantage of immunotherapeutics that rely on delivery of toxins or radionuclides is that these agents are active at all times. As such, there is a potential for damage to non-tumor cells and toxicity issues associated with immunotherapy. For example, cancer cells commonly shed surface-expressed antigens into the blood stream that are targeted by immunotherapeutics. As a result, many antibody-based therapies are diluted prior to reaching diseased tissue due to the interaction of the antibody with shed antigens rather than cancer cells thereby reducing the actual dose delivered to the diseased tissue.

For these and other related reasons, it is desirable to provide alternative and improved techniques for treating disease, particularly techniques that are less invasive and traumatic to the patient than existing techniques. It is also desirable to provide treatments that are effective only at targeted sites, such as diseased tissue, pathogens, or other undesirable matter in the body that minimize adverse side effects and improve efficacy. Further, it is desirable to provide techniques capable of being performed in a single or very few treatment sessions to facilitate patient compliance.

Thermotherapy may hold promise as a treatment for cancer and other diseases because it induces instantaneous necrosis (typically referred to as “thermo-ablation”) and/or a heat-shock response in cells (classical hyperthermia), leading to cell death via a series of biochemical changes within the cell. Because temperatures from about 40° C. to about 46° C. can cause irreversible damage to diseased cells, and healthy cells are capable of surviving exposure to temperatures up to around 46.5° C., elevating the temperature of cells in diseased tissue to between about 40° C. to about 46° C. may provide a treatment option that selectively destroys diseased cells while not causing damage to normal healthy tissues. Further, temperatures greater than 46° C. may be effective for the treatment of cancer and other diseases by causing an instantaneous thermo-ablative response. However, accurate and precise targeting is necessary to ensure that a minimal amount of healthy tissue is exposed to such temperatures.

Thermotherapy applied to a cell or diseased tissue in combination with ionizing radiation, such as, ultraviolet, x-ray, gamma, beta, alpha, neutron, or chemotherapy often results in an enhanced cytotoxic effect, which may be significantly greater than expected from an additive combination of the ionizing energy or chemotherapy doses. For example, a cell may exhibit a high level of susceptibility to an otherwise sub-lethal dose of either chemotherapeutic agent or ionizing radiation when that dose is combined with thermotherapy, even when the thermotherapy is also administered at sub-lethal dose. Such combination therapy has significant clinical potential because damaging side effects from a dose of either heat or ionizing radiation may be minimized or avoided.

The beneficial effects of thermotherapy may be further compounded by suitably targeting the thermotherapeutic agent (i.e., susceptors) to diseased cells, tissue or pathogen.

Some thermotherapy systems employ microwave or radio frequency (RF) hyperthermia, such as annular phased array systems (APAS), to tune energy for regional heating of deep-seated tumors in a patient. Such techniques are limited by heterogeneities of tissue electrical conductivities and that of highly perfused tissue. Typical problems include “hot spots” in healthy tissue with concomitant under-dosage in diseased tissue and difficulty in determining with adequate precision the heat dose delivered to a desired area. The latter precludes the development of prescriptive clinical protocols, which are necessary to ensure reproducible and predictable patient benefits following treatment. All of these factors make selective heating of specific regions with such thermotherapeutic systems very difficult.

### SUMMARY OF THE INVENTION

The invention described herein is directed to ligand conjugated particles, and in certain embodiments, the ligand conjugated particles may be thermotherapeutic agents.

The ligand conjugated particles of various embodiments may include an amino-functionalized nanoparticle forming a single magnetic domain; at least one linker in communication with the amino-functionalized nanoparticle; and at least one ligand coupled to the amino-functionalized nanoparticle or the linker. In some embodiments, the linker may be a bifunctional compound. In other embodiments, the linker may be a multi-subunit composition having one or more subunit selected from a haloalkyl, epoxide, vinyl heterocumulene, epoxypropene, polyethylene glycol, polypropylene or combination thereof. In still other embodiments, the linker may include one or more hydrophilic subunit, and in particular embodiments, the linker may be a mixture of chemically different compounds. For example, in certain embodiments, the linker may include at least one diepoxide, at least one poly(ethylene glycol) epoxyether, at least one poly(ethylene glycol) diglycidyl ether, at least one epichlorohydrin or combination thereof, and in some, the linker may include a mixture of epichlorohydrin and poly(ethylene glycol) diglycidyl ether.

The linker, of further embodiments, may include one or more terminal reactive group selected from amine, thiol, hydrazine, azide, disulphide, sulphonic acid, carboxylic acid, maleimide or combination thereof, and in other embodiments, the reactivity of terminal groups of the ligand conjugated particle may be based on substitution or addition chemistry. In particular embodiments, the carboxylic acid may be poly(ethylene glycol) ether based carboxylic acid; the azide may be 5-Azido-2 nitrobenzamide; the disulphide may be 3-(2-pyridyldithio)propionamide; and the maleimide may be 1,2-diacylethene or 3-maleimidylpropionamide.

The amino-functionalized particles of various embodiments may include substructures, said substructures comprising at least one linker, at least one ligand and at least one chelator or a combination thereof.

In certain embodiments, the ligand may be an antibody. In some embodiments, the ligand may be modified by incorporation of a group selected from a thiol or an amine, and in particular embodiments, the ligand may be modified with N-succinimidyl-5-acetylthioacetate.

According to some embodiments, the ligand conjugated particle may further comprise a biocompatible coating. In particular embodiments, the surface of the amino-functionalized nanoparticle forms the biocompatible coating.

The ligand conjugated particles of various embodiments may be a thermotherapeutic agent.

A ligand conjugated particle of other embodiments may include a functionalized magnetic nanoparticle and at least one linker in communication with the functionalized magnetic nanoparticle wherein the specific absorption rate (SAR) of said ligand conjugated nanoparticle is at least 5 fold higher than 20 nm Nanomag®-D-spio particles. In certain embodiments, the ligand conjugated particle further comprises a ligand coupled to the functionalized magnetic nanoparticle or the linker.

In various embodiments, a method of treating disease in a subject is provided comprising administering to the subject an effective amount of the ligand conjugated particle of aspects of the invention.

Other embodiments of the invention include a method for preparing a ligand conjugated particle including the steps of functionalizing a particle forming a single magnetic domain with amino or nitro groups, contacting the functionalized particle with a linker, and coupling a ligand to the particle or the linker to form a ligand conjugated particle. In certain embodiments, the functionalization step occurs at a pH of between about 7 and about 9. In other embodiments, the method for preparing a ligand conjugated particle includes the additional step of washing the ligand conjugated particle with an aqueous buffer solution. In some embodiments, the washing step occurs at a pH of between about 5 and about 8. In yet other embodiments, the method for preparing a ligand conjugated particle includes the step of sterilizing the ligand conjugated particle. According to some embodiments, the step of coupling the ligand to the particle or the linker to form the ligand conjugated particle occurs within 12 hours of the step of contacting the functionalized particle with the linker.

The ligand conjugated particles according to some embodiments range in size from 10-80 nm.

In various embodiments, a nanoparticle for thermotherapy prepared by a process comprising the steps of functionalizing a particle forming a single magnetic domain with amino or nitro groups, contacting the functionalized particle with a linker; and coupling a ligand to the particle or the linker to form a ligand conjugated particle is provided.

### BRIEF DESCRIPTION OF THE DRAWINGS

For a fuller understanding of the nature and advantages of the invention, reference should be made to the following detailed description taken in connection with the accompanying drawings, in which:

FIG. 1 illustrates a susceptor conjugate according to an embodiment of the invention;

FIG. 2 illustrates four synthesis strategies for preparing susceptor conjugates;

FIG. 3 is a size distribution graph for amino-functionalized particles (II) having diameters of 25 nm (black), 50 nm (dark gray) and 70 nm (light gray);

FIG. 4 is a graph depicting impedance spectroscopy data of the magnetic volume susceptibility at room temperature magnetic particles having a mean diameter of 70 nm at 200 Hz;

FIG. 5a is a schematic illustrating two possible ways a secondary goat anti-rabbit antibody can interact with a rabbit anti-goat antibody conjugated to the surface of a particle;

FIG. 5b is a schematic illustrating that a goat anti-mouse antibody can only interact with a rabbit anti-goat antibody conjugate when the rabbit anti-goat is in the proper orientation; and

FIG. 6 is a bar graph depicting the total bound antibody compared to the immunoreactivity per mg of iron with antibody conjugated particles prepared using the strategies illustrated in FIG. 2.

### DETAILED DESCRIPTION

This invention is not limited to the particular processes, compositions, or methodologies described, as these may vary. In addition, the terminology used in the description is for the purpose of describing the particular versions or embodiments only, and is not intended to limit the scope of the invention.

Unless defined otherwise, all technical and scientific terms used herein have the same meanings as commonly understood by one of ordinary skill in the art. Although any methods similar or equivalent to those described herein can be used in the practice or testing of embodiments of the invention, the preferred methods are now described.

All publications and references mentioned herein are incorporated by reference. Nothing herein is to be construed as an admission that the invention is not entitled to antedate such disclosure by virtue of prior invention.

It must be noted that, as used herein, and in the appended claims, the singular forms “a”, “an” and “the” include plural reference unless the context clearly dictates otherwise.

As used herein, the term “about” means plus or minus 10% of the numerical value of the number with which it is being used. Therefore, about 50% means in the range of 40%-60%.

“Administer”, as used herein in conjunction with the therapeutic nanoparticle compositions of the invention, means to administer a therapeutic directly into or onto a target tissue or to administer a therapeutic to a patient whereby the therapeutic impacts the tissue to which it is targeted. “Administering” a therapeutic may be accomplished by injection, infusion, or by either method in combination with other known techniques, to name a few. Such combination techniques include, but are not limited to, heating, radiation and ultrasound.

The term “alternating magnetic field” or “AMF”, as used herein, refers to a magnetic field that changes the direction of its field vector periodically, typically in a sinusoidal, triangular, rectangular or similarly shaped pattern, with a frequency in the range of from about 80% Hz to about 800 kHz. The AMF may also be added to a static magnetic field, such that only the AMF component of the resulting magnetic field vector changes direction. The AMF may be accompanied by an alternating electric field and may be electromagnetic in nature.

As used herein, the term “antibody” includes reference to an immunoglobulin molecule that is reactive with a particular antigen, and includes both polyclonal and monoclonal antibodies.

The term “diseased tissue”, as used herein, refers to tissue or cells associated with solid tumor cancers of any type, such as bone, lung, vascular, neuronal, colon, ovarian, breast and prostate cancer. The term diseased tissue may also refer to tissue or cells of the immune system, such as tissue or cells effected by AIDS; pathogen-borne diseases, which can be bacterial, viral, parasitic, or fungal, examples of pathogen-borne diseases include HIV, tuberculosis and malaria; hormone-related diseases, such as obesity; vascular system diseases; central nervous system diseases, such as multiple sclerosis; and undesirable matter, such as adverse angiogenesis, restenosis amyloidosis, toxins, reaction-by-products associated with organ transplants, and other abnormal cell or tissue growth.

An “effective amount” or “therapeutically effective amount” of a composition as used herein is a predetermined amount calculated to achieve the desired effect.

The term “energy source”, as used herein, refers to a device that is capable of delivering energy, of a form other than AMF, to a therapeutic for the purpose of activating a potentially radioactive source in the therapeutic.

The term “hyperthermia”, as used herein, refers to heating of tissue to temperatures between 40° C. and 60° C.

The term “impact” is used to convey a change in the appearance, form, characteristics and/or physical attributes of a tissue, cell or region of a patient to which a therapeutic is being provided, applied or administered.

The term “indication”, as used herein, refers to a medical condition or symptoms associated with a medical condition, such as cancer. For example, fatigue or fever may be an indication of subject in a diseased state.

The term “ligand” refers to a compound that specifically targets a molecule.

“Optional” or “optionally” may be taken to mean that the subsequently described structure, event or circumstance may or may not occur, and that the description includes instances where the event occurs and instances where it does not.

The term “target”, as used herein, refers to the material for which deactivation, rupture, disruption or destruction is desired. For example, diseased tissue may be a target for therapy.

Generally speaking, the term “tissue” refers to any collection of similarly specialized cells that are united in the performance of a particular function.

In its most basic form, the invention described herein is directed to ligand conjugated particles for use in thermotherapy. Various embodiments of the invention include a particle, at least one linker or cross-linking compound and at least one ligand.

The particles of various embodiments are magnetic particles and in other embodiments the particles are magnetic particles that are capable of generating heat when placed in an alternating magnetic field (AMF) or other energy source. Such particles may be referred to as magnetic energy susceptive particles or “susceptors” and may be useful for providing thermotherapy.

As used herein, the terms “susceptor” and “untargeted susceptor” refer to susceptors that have not been modified to interact with a specific cell type, molecule or other target. In contrast, “targeted susceptors”, “ligand conjugated” particles or susceptors and “susceptor conjugates” have been modified to interact with a specific target using, for example, an antibody that is covalently attached to the susceptor. Untargeted susceptors contain no such targeting mechanism.

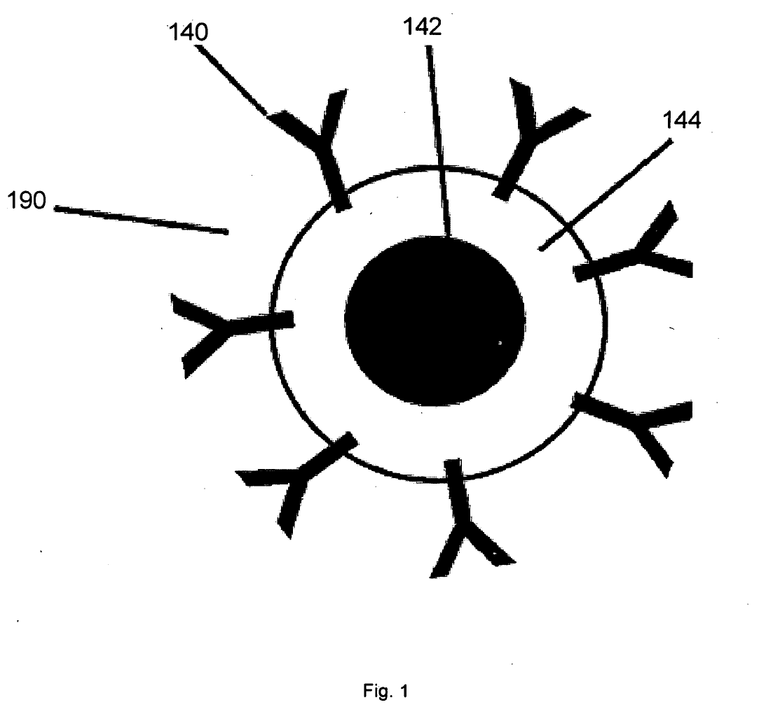

FIG. 1. illustrates a susceptor conjugate 100 according to an embodiment of the invention. A susceptor conjugate 100 comprises a magnetic energy susceptive particle or susceptor 142. The susceptor may include a coating 144 that fully or partially covers the susceptor 142. At least one targeting ligand 140 such as, but not limited to, an antibody may be located on an exterior portion of the susceptor 142. The targeting ligand 140 may be selected to seek out and attach to a specific target, such as a type of cell or diseased tissue. Heat is generated in the susceptor 142 when the susceptor 142 is exposed to an energy source, such as AMF. The coating 144 may enhance the heating properties of the susceptor 142, particularly if the coating 144 has a high viscosity, such as for example, a polymeric material.

Referring specifically to susceptors, in general, the heat generated by susceptors when placed in an AMF represents an energy loss as the magnetic material of the susceptor oscillates in response to the AMF. The amount of heat generated per cycle of magnetic field and the mechanism responsible for the energy loss depend on the specific characteristics of both the susceptor material and the magnetic field applied. In embodiments of the invention, the susceptor forms a single magnetic domain.

According to some aspects, the susceptor heats to a unique temperature, referred to as the Curie temperature, when subjected to an AMF or other energy source. The Curie temperature is the temperature at which a reversible ferromagnetic to paramagnetic transition of the magnetic material of the susceptor occurs. Below this temperature, the magnetic material generates heat in an applied AMF, but above the Curie temperature, the magnetic material is paramagnetic and magnetic domains are unresponsive to AMF. Thus, the magnetic material does not generate heat when exposed to AMF above the Curie temperature. As the magnetic material cools to a temperature below the Curie temperature, the material recovers its magnetic properties and resumes heating when AMF is applied. This cycle may be repeated continuously during exposure to the AMF. As such, the magnetic material of the susceptor is able to self-regulate heating temperature. In embodiments of the invention, the magnetic material may be selected to possess a Curie temperature between about 40° C. and about 150° C.

The temperature to which a susceptor heats is dependent upon the magnetic properties of the susceptor material, characteristics of the magnetic field, and the cooling capacity of the target site, among other factors. Selection of the susceptor material and AMF characteristics may be tailored to optimize treatment efficacy for a particular target type. Many aspects of the susceptor, such as material composition, size, and shape, directly affect heating properties and these characteristics may be tailored to achieve desired heating properties. For example, the size of the susceptor utilized in thermotherapy may depend upon the particular application for which the susceptor will be used (i.e., the temperature to be achieved) and on the material that comprises the susceptor.

The size of the susceptor may also determine the total size of the “susceptor conjugate” including, for example, a linker and a ligand. In various embodiments, the size of susceptor is from about 0.1 nm to about 250 nm. The susceptor conjugate size may also depend upon the indication, the materials that comprise susceptor and susceptor conjugate, administration route, and the method of use. In some embodiments, it may be desired that the susceptor or susceptor conjugate administered to a patient, for example, via intravenous injection, avoid uptake by the reticuloendothelial system (RES) and subsequent distribution to the liver, spleen, lungs, kidneys, heart and bone marrow in order to achieve increased therapeutic composition concentration and long residence time in the bloodstream. To successfully avoid uptake by the RES, the diameter of the susceptor or susceptor conjugate may be less than about 30 nm, and in particular, in embodiments in which the susceptor contains magnetite (Fe₃O₄), the diameter of the susceptor conjugate may be between about 8 nm and about 20 nm. The susceptors of such susceptor conjugates retain a sufficient magnetic moment for heating in an applied AMF, while allowing the therapeutic composition to evade uptake by the RES. In some embodiments, ferromagnetic susceptors having a diameter larger than about 8 nm may be appropriate for thermotherapeutic applications. In other embodiments, other elements, such as, for example, cobalt are included in the magnetite susceptor. The inclusion of secondary elements may allow the size range of the susceptor and the susceptor conjugate comprising such susceptor to be smaller. For example, cobalt, although smaller that magnetite, generally possesses a larger magnetic moment than magnetite, which may contribute to the overall magnetic moment of cobalt-containing magnetite susceptor while decreasing the size of the therapeutic composition.

Susceptors for use in embodiments of the invention may include any number of materials that provide an appropriate magnetic moment and size. The material composition of a susceptor may be selected based on the particular target. For example, susceptors in embodiments include, but are not limited to, iron oxide particles and FeCo/SiO₂ particles. Because the self-limiting Curie temperature is directly related to the susceptor material as is the total heat delivered to the target, susceptor compositions may be designed for different target types. Such tuning may be required to achieve desired heating of each target type given the unique heating and cooling capacities based on the target's composition and location within the patient's body. For example, a tumor located within a region of a patient that is poorly supplied by blood and that is relatively insulated may require a lower Curie temperature susceptor material for effective thermotherapy than a tumor located near a major blood vessel. Targets located in the bloodstream will require susceptor materials with specific Curie temperatures as well. As such, in addition to magnetite, susceptors of various embodiments may include, for example, cobalt, iron, rare earth metals and the like and combinations thereof.

The specific absorption rate (SAR) may also be considered in selecting susceptor material. The SAR for a given material is generally described as the rate at which the material absorbs radio frequency (RF) energy when exposed to a RF electromagnetic field. For example, series EMG700 and EMG1111 iron oxide particles of about 110 nm diameter available from Ferrotec Corp. (Nashua, N.H.) have an SAR of about 310 Watts per gram of particle at 1,300 Oerstedt flux-density and 150 kHz frequency. Other particles, such as the FeCo/SiO₂ particles available from Inframat Corp. (Willington, Conn.) have an SAR of about 400 Watts per gram of particle under the same magnetic field conditions.

In some embodiments, the susceptors include a coating. Suitable coating materials may include, but are not limited to, synthetic and biological polymers, copolymers and polymer blends, and inorganic materials. In embodiments utilizing synthetic polymers as coating materials, such coatings may include, for example, acrylate, siloxane, styrene, acetate, alkylene glycol, alkylene, alkylene oxide, parylene, lactic acid, glycolic acid, and combinations thereof, and in certain embodiments, such coatings may include a hydrogel polymer, a histidine-containing polymer, and a combination of a hydrogel polymer and a histidine-containing polymer. Further embodiments encompass coating materials that include biological materials such as, for example, polysaccharides, polyaminoacids, proteins, lipids, glycerols, fatty acids, and combinations thereof, and in other embodiments, biological materials for use as a coating material may include heparin, heparin sulfate, chondroitin sulfate, chitin, chitosan, cellulose, dextran, alginate, starch, carbohydrate, and glycosaminoglycan. In embodiments utilizing a protein coating material, such proteins may include extracellular matrix proteins, proteoglycans, glycoproteins, albumins, and gelatin. In still other embodiments, biological coating materials are used in combination with suitable synthetic polymer materials.

Other embodiments of the invention include susceptors having an inorganic coating material such as, but not limited to, metals, metal alloys, and ceramics, such as, for example, hydroxyapatite, silicon carbide, carboxylate, sulfonate, phosphate, ferrite, phosphonate, and oxides of Group IV elements of the Periodic Table of the Elements. In other embodiments, the inorganic coating materials are a component of a composite coating that also contains biological or synthetic polymers.

In some embodiments, where the susceptor is formed from a magnetic material that is biocompatible, the surface of the susceptor itself may act as the biocompatible coating. In other embodiments, the coating material may serve as to make the susceptor biocompatible. In still other embodiments, the coating material may facilitate transport of susceptor into a cell by, for example, transfection. Such coating materials, referred to as transfection agents, may include, for example, a vector, such as a plasmid, virus, phage, viron or a viral coat, prion, polyaminoacid, cationic liposome, amphiphile, non-liposomal lipid, or any combination thereof. In other embodiments, a biocompatible coating material may be a composite of transfection agent(s) with organic and/or inorganic materials. In such embodiments, the combination of coating materials and/or transfection agents may be tailored for a particular type of cell or diseased tissue and a specific location within a patient's body.

In some embodiments of the invention, a linker is coupled to the susceptor or susceptor coating. The linker, in some embodiments, may be a bifunctional compound that contacts and binds to an outer surface of the susceptor while covalently binding to a ligand thereby attaching the ligand to the outer surface of the susceptor. In other embodiments, the linker binds to the outer surface of a susceptor and facilitates evasion of the susceptor from the reticuloendothelial system (RES). For example, functionalization of a susceptor with a polyethylene glycol (PEG) linker through a process known in the art as “pegylation” is effective in avoiding detection and uptake by the RES and facilitates penetration of the altered vasculature of tumors via the enhanced permeability and retention (EPR) effect, resulting in preferential accumulation of susceptors in tumor interstitium.

Depending upon whether the linker is short or long, rigid or flexible, hydrophobic or hydrophilic, the linker can affect the properties of the final conjugates. Linkers of various embodiments include a hydrophobic or hydrophilic organic, inorganic or a mixture of chemically different compounds. In some embodiments, the linker may include an alkyl, alkene, alkyne, haloalkyl, epoxide, vinyl, or heterocumulene compound, and in other embodiments, the linker may include a multi-subunit compound. The linkers of such embodiments are not limited by the number and/or type of subunits in a multi-subunit compound and may include subunits of, for example, epoxypropene, polyethylene glycol, polypropylene glycol, and the like and combinations thereof. In still other embodiments, the linkers include one or more epichlorohydrin, diepoxide or combinations thereof. Examples of linkers that are encompassed by embodiments of the invention include, but are not limited to, poly(ethylene glycol) epoxyether, poly(ethylene glycol) diglycidyl ether, and a mixture of epichlorohydrin and poly(ethylene glycol) diglycidyl ether.

Linkers of various embodiments further include one or more terminal reactive groups. The type of terminal reactive group may vary depending on the type of reaction chemistry required to couple a susceptor of a particular type to a certain type of ligand, form a covalent bond with a certain ligand or otherwise bind a susceptor to such ligand. In certain embodiments, the reactivity of the terminal group may be based on substitution or addition chemistry. Exemplary terminal reactive groups may include, but are not limited to, carboxylic acids, amines, hydrazines, azides, thiols, disulphides, sulphonic acid, vinyl, 1,2-diacylethene and derivatives and combinations thereof, and in particular embodiments, the terminal reactive group may be an amine, thiol or carboxylic acid moiety. In some embodiments, the carboxylic acid terminal group may be a poly(ethylene glycol) ether based carboxylic acid, the azide terminal group may be a 5-azido-2-nitrobenzamide, the disulfide terminal group may be a 3-(2-pyridylithio)propionamide, and the 1,2-diacylethene terminal group may be a maleimide or a 3-maleimidylpropionamide.

Ligands of embodiments of the invention may be selected to ensure that the susceptor selectively attaches to, or otherwise associates with, the selected target. In some embodiments, ligands allow the targeting of cancer or disease markers on cells. In other embodiments, ligands facilitate the targeting of a specific type of biological matter in a patient. Various embodiments include ligands, such as, but not limited to, proteins, peptides, antibodies, antibody fragments, saccharides, carbohydrates, glycans, cytokines, chemokines, nucleotides, lectins, lipids, receptors, steroids, neurotransmitters, Cluster Designation/Differentiation (CD) markers, imprinted polymers, and combinations thereof. In certain embodiments, protein ligands include, for example, cell surface proteins, membrane proteins, proteoglycans, glycoproteins, peptides, and the like; nucleotide ligands include, for example, single-stranded nucleotides, double stranded nucleotides, complimentary nucleotides, and polynucleotide fragments; and lipid ligands may include, for example, phospholipids, glycolipids, and the like. In other embodiments, the ligand is an antibody or antibody fragment.

Antibodies useful in embodiments of the invention are not limited by a particular type of antibody. Antibodies useful in some embodiments may be genetically engineered, such as for example, chimeric antibodies (e.g., humanized murine antibodies) and heteroconjugate antibodies (e.g., bispecific antibodies). Antibodies may also include antigen binding forms of antibodies, including fragments with antigen-binding capability, such as, Fab′, F(ab′)₂, Fab, Fv and rIgG, and recombinant single chain Fv fragments (scFv). Such fragments are well known in the art and can be found, for example, in Pierce Catalog and Handbook, 1994-1995 (Pierce Chemical Co., Rockford, Ill. and Kuby, J., Immunology, 3.sup.rd Ed., W.H. Freeman & Co., New York (1998). Antibodies of other embodiments encompass bivalent or bispecific molecules such as, but not limited to, those described in Kostelny et al. J. Imminol. 148:1547 (1992), Pack and Pluckthun Biochemistry 31:1579 (1992), Hollinger et al. supra, (1993), Gruber et al. J. Immunol. 5368 (1994), Zhu et al. Protein Sci 6:781 (1997), Hu et al. Cancer Res. 56:3055 (1996), Adams et al. Cancer Res. 53:4026 (1993), and McCartney et al. Protein Eng. 8:301 (1995).

Ligands of embodiments of the invention may be prepared to adhere to any marker or antigen known in the art. The choice of a marker may vary depending on the selected target, but in general, markers that may be useful in embodiments of the invention include, but are not limited to, cell surface markers, a member of the vascular endothelial growth factor receptor (VEGFR) family, a member of carcinoembryonic antigen (CEA) family, a type of anti-idiotypic mAB, a type of ganglioside mimic, a cluster designation/differentiation (CD) antigen, a member of the epidermal growth factor receptor (EGFR) family, a type of a cellular adhesion molecule, a member of the MUC-type mucin family, a cancer antigen (CA), a matrix metalloproteinase, a glycoprotein antigen, a melanoma associated antigen (MAA), a proteolytic enzyme, a calmodulin, a member of tumor necrosis factor (TNF) receptor family, an angiogenesis marker, a melanoma antigen recognized by T cells (MART) antigen, a member of the melanoma antigen encoding gene (MAGE) family, a prostate membrane specific antigen (PMSA), a small cell lung carcinoma antigen (SCLCA), a T/Tn antigen, a hormone receptor, a tumor suppressor gene antigen, a cell cycle regulator antigen, an oncogene antigen, an oncogene receptor antigen, a proliferation marker, a proteinase involved in degradation of extracellular matrix, a malignant transformation related factor, an apoptosis-related factor, and a human carcinoma antigen. For example, specific markers for breast cancer may be chosen from cell surface antigens such as, but not limited to, members of the MUC-type mucin family, an epithelial growth factor (EGFR) receptor, a carcinoembryonic antigen (CEA), a human carcinoma antigen, a vascular endothelial growth factor (VEGF) antigen, a melanoma antigen (MAGE), family antigen, a T/Tn antigen, a hormone receptor, growth factor receptors, a cluster designation/differentiation (CD) antigen, a tumor suppressor gene-product, a cell cycle regulator, an oncogene-product, an oncogene receptor, a proliferation marker, an adhesion molecule, a proteinase involved in degradation of extracellular matrix, a malignant transformation related factor, an apoptosis related factor, a human carcinoma antigen, glycoprotein antigens, DF3, 4F2, MGFM antigens, breast tumor antigen CA 15-3, calponin, cathepsin, CD 31 antigen, proliferating cell nuclear antigen 10 (PC 10), and pS2.

In another embodiment, ligands may be targeted to an antigen associated with a disease of a patient's immune system. For example, the marker or markers to which the ligand is targeted may be selected such to include viable targets on, for instance, T cells or B cells, or the ligand may have an affinity for a target associated with an immune system disease such as, for example, a protein, a cytokine, a chemokine, an infectious organism, and the like.

In yet another embodiment, ligands may be targeted to an antigen associated with a pathogen-borne condition. In general, a pathogen may include any disease-producing agent such as, for example, a bacterium, a virus, a microorganism, a fungus, a parasite, or a prion. The ligands of this embodiment may have an affinity for a cell marker or markers associated with a pathogen or for a marker or markers that represent a target on infected cells. For example, the ligand may be selected to target the pathogen itself, such as a bacterium, including, but not limited to, Escherichia coli or Bacillus anthracis; a virus, including, but not limited to, Cytomegalovirus (CMV), Epstein-Barr virus (EBV), hepatitis virus, such as, Hepatitis B virus, human immunodeficiency virus, such as HIV, HIV-1 or HIV-2, or a herpes virus, such as Herpes virus 6; a parasite, including, but not limited to, Trypanasoma cruzi, Kinetoplastid, Schistosoma mansoni, Schistosoma japonicum or Schistosoma brucei; or a fungus condition, including, but not limited to, Aspergillus, Cryptococcus neoformans or Rhizomucor.

In particular embodiments of the invention, modified antibodies can be produced by reacting an antibody or antibody fragment with a modifying agent. For example, organic moieties can be bonded to the antibody in a non-site specific manner by employing an amine-reactive modifying agent, for example, N-hydroxysuccinimide (NHS). In other embodiments of the invention, modified human antibodies or antigen-binding fragments are prepared by reducing disulfide bonds (e.g., intra-chain disulfide bonds) of an antibody or antigen-binding fragment. The reduced antibody or antigen-binding fragment is then reacted with a thiol-reactive modifying agent to covalently bond the antibody to the linker. Modified human antibodies and antigen-binding fragments of aspects of the invention comprising an organic moiety that is bonded to specific sites of an antibody can be prepared using suitable methods, such as reverse proteolysis (Fisch et al. Bioconjugate Chem. 3:147 153 (1992); Werlen et al. Bioconjugate Chem. 5:411 417 (1994); Kumaran et al., Protein Sci. 6(10):2233 2241 (1997); Itoh et al., Bioorg. Chem., 24(1): 59 68 (1996); Capellas et al., Biotechnol. Bioeng., 56(4):456 463 (1997)) and the methods described in Hermanson Bioconjugate Techniques, Academic Press: San Diego, Calif. (1996).

In yet another embodiment, ligands may be targeted to cells or tissue associated with an undesirable condition. Such undesirable conditions may be associated with a disease, but may also be present in normal conditions. The ligand may be targeted directly to a protein or other antigen that is associated with the undesirable condition or to another molecule associated with a biological molecular pathway related to the undesirable condition. For example, apolipoprotein B on low density lipoprotein (LDL) may be used as a target molecule to treat arteriosclerosis, or a gastric inhibitory polypeptide receptor may be targeted to treat obesity. Further examples include ligands directed to targets associated with hormone-related disease wherein the target is the hormone itself or a hormone or signaling peptide associated with the hormone's production, and non-cancerous diseased tissue wherein the target is an antigen or peptide associated with the diseased tissue or a protein or peptide associated with the deposition of the non-cancerous diseased tissue.

In further embodiments, ligands may be targeted antigens or proteins associated with organ rejection following an organ transplant. Targets in such embodiments may vary depending on the particular type of organ transplanted and may, for example, include immune cells such as T cells or B cells.

In still further embodiments, the ligand may be targeted to a toxin in a patient. Such toxins include any poison produced by an organism such as, but not limited to, bacterial toxins, plant toxins, insect toxin, animal toxins, and man-made toxins.

Further examples of ligands of embodiments of the invention can be found in U.S. patent application Ser. No. 10/696,399, which is hereby incorporated by reference in its entirety.

The ligand in certain embodiments may be covalently bonded to the susceptor, a coating associated with the susceptor or a linker bound to a surface of the susceptor. In some embodiments, the ligand may be modified to enhance the ability of the ligand to covalently bond to the linker or coating and embodiments are not limited by the type of modification. For example, in certain embodiments, the ligand may be thiol-modified. Methods for preparing modified ligands are well known in the art and are commercially available.

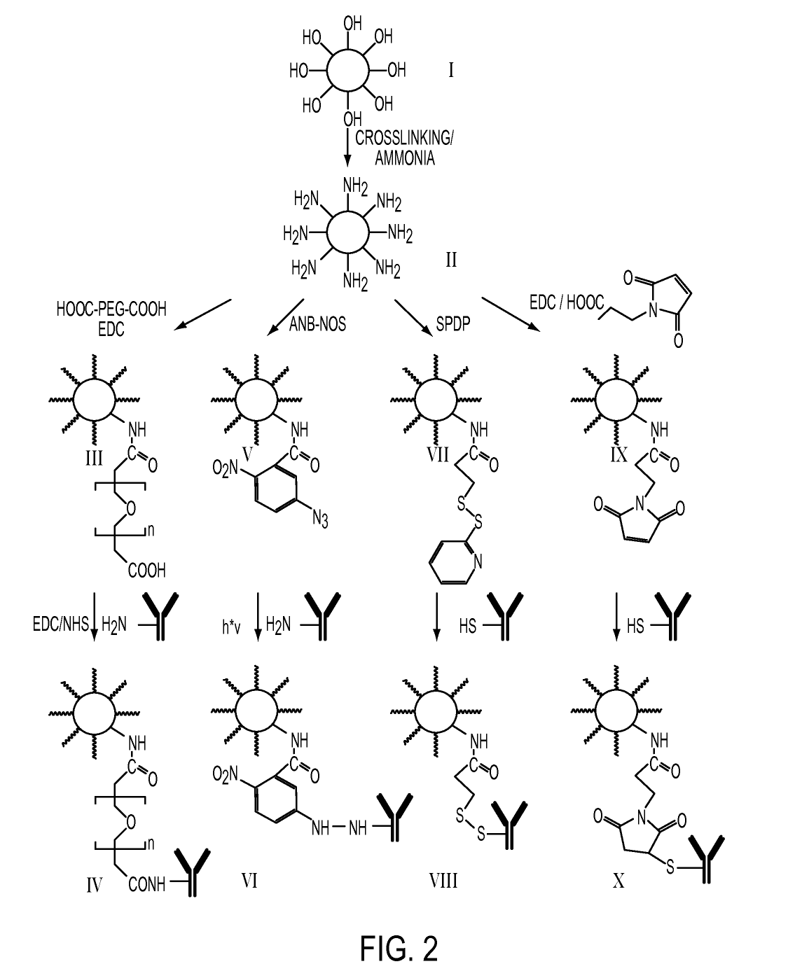

Further embodiments of the invention include methods for preparing a ligand conjugated particle or “susceptor conjugate.” A schematic of various methods for preparing a susceptor conjugate is provided in FIG. 2, and such methods may include the steps of preparing particles (I), amino-functionalizing the particles (II), reacting a linker, such as those described above, with the amino-functionalized particles (II) to form, for example, particles (III, V, VII and XI), and reacting a second functional group on the linker with the ligand to form ligand conjugated particles or susceptor conjugates (IV, VI, VIII and X). Various modifications, known in the art, may be made to any step of the schemes provided in FIG. 2 and described herein, or one or more steps may be substituted for another equivalent step. Such modifications are encompassed within the scope of the invention. For example, particles or “susceptors” may be functionalized with a thiol or other reactive group, or various steps may be combined such that more than one type of linker or ligand is coupled to the susceptor. In other embodiments of the invention, the susceptors are optimized with a specific ratio of conjugated to non-conjugated surface area, such that an effective amount of linker or ligand is associated with the susceptor for treatment of a disease. In further embodiments, the steps of washing and/or sterilizing the susceptor conjugates are included in the method. The washing and/or sterilization step may include microfiltration or other such methods known in the art.

Other embodiments of the invention include a method for preparing a ligand conjugated particle including the steps of functionalizing a particle forming a single magnetic domain with amino or nitro groups, contacting the functionalized particle with a linker, and coupling a ligand to the particle or the linker to form a ligand conjugated particle.

Still other embodiments include methods for treating a disease using the susceptors and susceptor conjugates of the invention. The diseases that may be treated using susceptor conjugates encompass a broad range of diseases and are only limited by the availability of a marker for the disease. Use of the untargeted susceptors is not limited by the availability of such a marker. Thus, any disease currently known or discovered in the future is encompassed by the methods of embodiments. For example, in one embodiment, susceptor conjugates wherein ligands are targeted to cancer cells are administered to a patient, the susceptor conjugates become attached to or become associated with the cancer cells, and the patient is exposed to an alternating magnetic field (AMF), Heat generated by the susceptors as a result of the AMF destroys (i.e., induces apoptosis) or otherwise deactivates the cancer cells immediately or over time. In addition, the heat generated by the susceptors and/or apoptosis may stimulate the production and release of heat shock proteins, such as, for example, HSP 70, the presence of which can stimulate an immune reaction against any remaining cancer cells. Such a stimulated immune response may also serve to protect the individual from future developments of cancer and other disease.

In other embodiments of the invention, modified surface charge and particle size may contribute to the effectiveness of the susceptors and susceptor conjugates in the treatment of disease. For example, in one aspect of the invention, susceptor surface charge is modified to reduce clearance of the nanoparticles. In one embodiment of the invention, a substantial portion of the susceptor surface is functionalized to provide a desired surface charge and zeta potential. In another embodiment, blocking agents that allow for selective functionalization of the susceptor surface, such as, for example, sulfo-NHS-acetate, are used to fine tune surface charge and zeta potential.

The surface chemistry or porosity of the susceptor or ligands of the susceptor conjugates may also be tailored such that the susceptors or susceptor conjugates remain external to target cells or, alternatively, are internalized into the target cells. Once associated with the target cells either externally or internally, the susceptors or susceptor conjugates may be energized as AMF energy is absorbed, and may, for example, heat up, and the heat generated may pass through the coating or linker and through interstitial regions to the target cell by, for example, convection, conduction, radiation, or a combination of heat transfer mechanisms. The target cells exposed to heat may become damaged, and in particular embodiments, such target cells may become damaged to the extent that the damage is irreparable. In certain embodiments, the target cells die via necrosis, apoptosis, or another mechanism when a sufficient amount of energy is transferred to the target cells.

The amount of susceptor or susceptor conjugate administered to a patient may vary and may depend the disease being treated, and the location of the diseased tissue. Moreover, the dosage may vary depending on the mode of administration. For example, a lower dosage may be required if the susceptor or susceptor conjugate is administered locally to, for instance, into or the area near a tumor, or systemically. The dosage to be administered may further depend on the characteristics of the subject being treated (e.g., age, weight, sex, health, types of concurrent treatment, if any, and frequency of treatments). Provided such information, it is within the purview of the skilled artisan (e.g. clinician) to determine the amount of susceptor or susceptor conjugate that would constitute a therapeutically effective amount. The selection of the specific route of administration and the dose regimen may be adjusted or titrated by such clinician according to methods known in the art in order to obtain an optimal clinical response.

Various routes of administration are contemplated in embodiments of the invention and the susceptors and susceptor conjugates can be administered in the conventional manner by any route where they are active. For example, administration can be, but is not limited to, systemic, parenteral, subcutaneous, intravenous, intramuscular, intraperitoneal, topical, transdermal, oral, buccal, or ocular routes, or intravaginally, by inhalation, by depot injections, or by implants. Modes of administration for susceptors and susceptor conjugates of certain embodiments of the invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams. Further, in some embodiments, methods of administration may include, but are not limited to, intravascular injection, intravenous injection, intraperitoneal injection, subcutaneous injection, and intramuscular injection. In other embodiments, susceptor or susceptor conjugates may be administered using perisurgical administration techniques including, but not limited to, a wash, lavage, as a rinse with sponge, or other surgical cloth. In other embodiments, routes of administration include injection or infusion in combination with other known techniques including, but not limited to, heating, radiation and ultrasound.

Methods of certain embodiments of the invention may further include formulating susceptors or susceptor conjugates in a pharmaceutical composition with a pharmaceutically acceptable excipient, vehicle or carrier. For example, in one embodiment, a pharmaceutical composition is prepared by dispersing or isolating susceptors or susceptor conjugates, admixing the susceptors or susceptor conjugates with a pharmaceutically acceptable excipient, vehicle or carrier, and, optionally, other ingredients to formulate pharmaceutical composition.

Pharmaceutical formulations containing susceptors and susceptor conjugates of aspects of the invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's ‘The Pharmaceutical Basis of Therapeutics, 6th Edition, MacMillan Publishing Co., New York (1980) can be consulted.

Referring specifically to parenteral administration routes, the susceptors and susceptor conjugates in certain embodiments of the invention can be formulated for parenteral administration by injection (e.g., by bolus injection or continuous infusion). The susceptors and susceptor conjugates may be administered by continuous infusion subcutaneously over a period of about 15 minutes to about 24 hours. Formulations for injection can be presented in unit dosage form (e.g., in ampoules or in multi-dose containers) with an added preservative. The formulations can take such forms as suspensions, solutions or emulsions in oily or aqueous vehicles, and can contain formulatory agents such as suspending, stabilizing and/or dispersing agents.

For oral administration, the susceptors and susceptor conjugates can be formulated by combining these compounds with pharmaceutically acceptable carriers known in the art. Such carriers enable the susceptors and susceptor conjugates to be formulated as tablets, pills, dragees, capsules, liquids, gels, syrups, slurries, suspensions and the like, for oral ingestion by a patient. Pharmaceutical formulations for oral administration can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol and cellulose preparations such as, but not limited to, maize starch, wheat starch, rice starch, potato starch, gelatin, gum tragacanth, methyl cellulose, hydroxypropylmethyl-cellulose, sodium carboxymethyl-cellulose, and polyvinylpyrrolidone (PVP). If desired, disintegrating agents can be added, such as, but not limited to, cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical formulations which can be used orally may further include, but are not limited to, push-fit capsules made of gelatin and soft, sealed capsules made of gelatin and a plasticizer, such as glycerol or sorbitol. The push-fit capsules can contain susceptors or susceptor conjugates in admixture with filler such as, for example, lactose, binders such as starches, and/or lubricants such as, talc or magnesium stearate and, optionally, stabilizers. In soft capsules, the susceptors or susceptor conjugates may be dissolved or suspended in suitable liquids, such as fatty oils, liquid paraffin, or liquid polyethylene glycols. In addition, stabilizers may optionally be added. All formulations for oral administration should be in dosages suitable for such mode of administration.

In buccal administration routes, the susceptors or susceptor conjugates may be formulated as tablets or lozenges using conventional techniques known in the art.

For administration by inhalation, susceptors or susceptor conjugates are delivered in the form of an aerosol spray or mist from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol administration, the dosage unit can be determined by providing a valve to deliver a metered amount. Capsules and cartridges of gelatin, for example, for use in an inhaler or insufflator can be formulated containing a powder mix of the susceptors or susceptor conjugates and a suitable powder base such as lactose or starch.

The susceptors or susceptor conjugates in other aspects of the invention can be formulated in rectal compositions such as suppositories or retention enemas, such as those, for example, containing conventional suppository bases such as cocoa butter or other glycerides.

The susceptors or susceptor conjugates can also be formulated as a depot preparation. Such long acting formulations are administered in one embodiment by implantation (e.g., subcutaneously or intramuscularly) or by intramuscular injection. Depot injections can be administered at about 1 to about 6 months or longer intervals. As such, the susceptors or susceptor conjugates are formulated with suitable polymeric or hydrophobic materials (e.g., as an emulsion in an acceptable oil) or ion exchange resins, or as sparingly soluble derivatives, for example, as a sparingly soluble salt to facilitate extended and controlled release.

In transdermal administration routes, the susceptors or susceptor conjugates of certain embodiments of the invention can be applied to a plaster or other transdermal therapeutic system known in the art.

Pharmaceutical compositions of susceptors or susceptor conjugates may further comprise suitable solid or gel phase carriers or excipients. Examples of such carriers or excipients include, but are not limited to, calcium carbonate, calcium phosphate, silica, sugar, starch, cellulose derivatives, gelatin, and polymers (e.g., polyethylene glycol (PEG), polylactic acid (PLA), poly(D,L-glycolide) (PLG), poly(lactide-co-glycolide) (PLGA), and poly(cyanoacrylate) (PCA)).

The susceptors and susceptor conjugates of aspects of the invention may be administered in combination with other active ingredients, such as, for example, adjuvants, protease inhibitors, or other compatible drugs or compounds where such combination is seen to be desirable or advantageous in achieving the desired effects of the methods described herein.

Once administered to a patient, delivery of susceptors or susceptor conjugates to the target may be enhanced by applying a static magnetic field to the patient in a region of the diseased tissue based on the magnetic character of the particles.

### EXAMPLES

In order that the invention disclosed herein may be more efficiently understood, examples are provided below. It should be understood that these examples are for illustrative purposes only and are not to be construed as limiting the invention in any manner.

### Example 1

### Preparation and Characterization of BNF Susceptors

Bionized nanoferrite (BNF) susceptors were fabricated by high-pressure homogenization (HPH) according to the core-shell method described in Grüttner et al. J. Magn. Magn. Mater. 311:181 (2006). A monodisperse aqueous iron oxide suspension (25 mg/ml) was homogenized with an excess of dextran at pressures above 500 bar and at temperatures above 70° C. for 30 min. BNF susceptors with an iron content of greater than 50% (w/w) were obtained after magnetic sedimentation in a crystallization disk at a NdFeB permanent magnet and washing with deionized water. The iron content of the susceptors was determined by gravimetric measurement of particle concentration and spectrophotometric measurement of the iron concentration of the particle suspension (Spectroquant®-Kit, Merck) after decomposition of the iron oxide with concentrated hydrochloric acid.

The BNF susceptors were crosslinked using a modified Josephson method with a mixture of poly(ethylene glycol) diglycidyl ether, MW=526 (Aldrich) and epichlorohydrin (ACROS) at pH 11-12 for 24 h at room temperature. After magnetic separation and washing with deionized water, a BNF susceptor suspension with an iron concentration of 20-25 mg/ml was obtained. The susceptors were functionalized with amino groups by shaking with ammonia at room temperature for 24 h. The BNF susceptors were washed three times with deionized water by magnetic separation and filtered through 0.22 mm Millex-GP filters (Millipore).

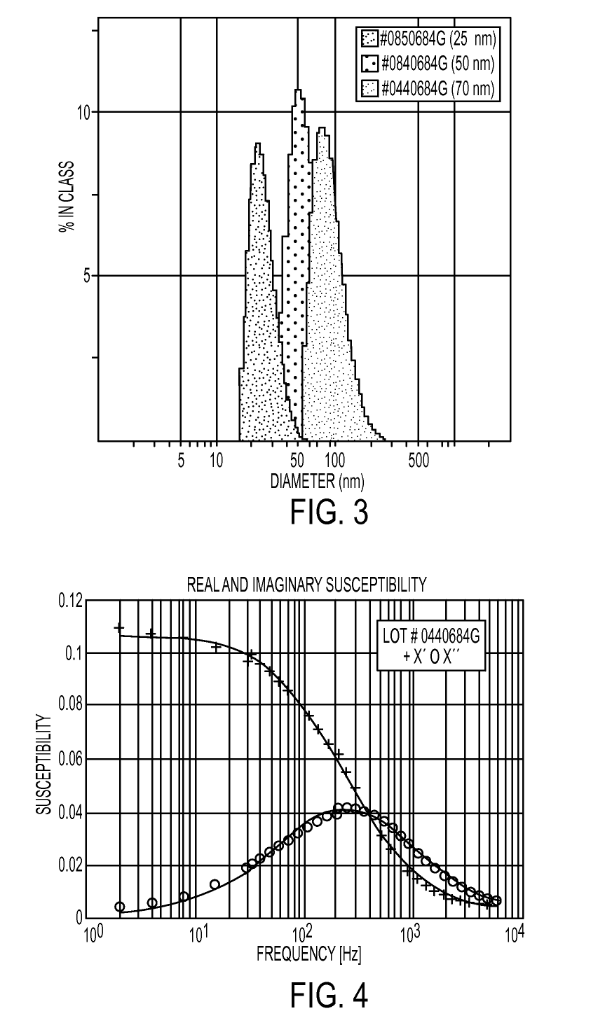

BNF susceptors having narrow particle size distributions were obtained in three different diameter ranges of 70-100, 40-70 and 20-40 nm, respectively, depending upon preparation conditions. Crosslinking and amination did not influence the initial particle diameters. FIG. 3 shows the size distributions of three lots of crosslinked and amino-functionalized BNF susceptors (FIG. 2 II) with mean diameters of 25 nm (#0850684G), 50 nm (#0840684G) and 70 nm (#0440684G), as measured by photon correlation spectroscopy (PCS).

A preliminary assessment of the magnetic properties of the BNF susceptor suspension was obtained by measuring the frequency dependence of the volume susceptibility in a magnetic field with impedance spectroscopy (IS). For magnetic characterization the volume susceptibility of BNF susceptors was measured in dependence on the frequency of the magnetic field by IS at amplitudes of the external magnetic field ranging from 0.1 to 0.5 mT. FIG. 4 depicts impedance spectroscopy data of the magnetic volume susceptibility at room temperature magnetic particles having a mean diameter of 70 nm at about 200 Hz (#0440684G). The resonance peak of the imaginary part of the susceptibility at room temperature of the BNF susceptor lot #0440684G at about 200 Hz was in accordance with expectations related to a dominating Brownian relaxation. The corresponding low-frequency real susceptibility has a value of 0.115 due to the high magnetite content of the susceptor suspension. These results suggest that BNF susceptors contain a significant fraction of thermally blocked single-domain particles at these frequencies and at room temperature.

The specific absorption rate (SAR) of the BNF susceptors was determined in a suitably modified AMF calorimeter by inducing the particles to heat with an AMF having a fixed frequency of 15371 kHz and varying flux densities. As shown in Table 1, SAR values for each susceptor type were calculated from the rate of rise of temperature measured in the water when the particle suspension was heated by the AMF generated in a solenoid coil. The SAR values were corrected for thermal properties of the calorimeter and coil using the appropriate reference blank, water or phosphate buffered saline (PBS) and were normalized by iron content. As further shown in Table 1, the SAR data of BNF susceptors are 6-7 fold higher than the corresponding data of 20 nm Nanomag®-D-spio particles. These results suggest that a significant increase in tumor response should occur when effective concentrations of BNF susceptors linked to anti-tumor monoclonal antibodies reach cancer cells and when they are induced to heat by an external AMF source.

TABLE 1
SAR Data for BNF Susceptors as a Function
of the Amplitude of the AMF

| Susceptor Type | SAR (W/g iron) | | | |
|---|---|---|---|---|
| (diameter) | 29 kA/m | 58 kA/m | 86 kA/m | 104 kA/m |
| BNF-1 (90 nm) | 140 | 437 | 528 | 642 |
| BNF-2 (60 nm) | 131 | 389 | 476 | 523 |
| BNF-3 (30 nm) | 90 | 253 | 380 | 438 |
| Nanomag ®-D-spio | 27 | 66 | 76 | 105 |
| (20 nm) | | | | |

(Nanomag ®-D-spio SAR data provided for comparison)

### Example 2

### Covalent Antibody Binding on the Surface of BNF Susceptors

Various strategies to covalently bind a model antibody, rabbit anti-goat IgG, to the BNF susceptors were evaluated using an immunoassay with goat anti-rabbit IgG-horse raddish peroxidase (HRP) and goat anti-mouse IgG-HRP. Antibody binding strategies, which are based on the reaction with amino groups of the antibody molecule, were compared with those that require sulfhydryl-labeled antibodies.

### Example 3

### Synthesis of BNF Susceptors with Polyethylene Glycol COOH Groups on the Surface and Conjugation with Rabbit Anti-Goat IgG

Five milligrams (26 μmol) N-ethyl-N′-(3-dimethylaminopropyl)carbodiimide hydrochloride (EDC) and 14 μl (26 μmol) polyethylene glycol 600 diacid were dissolved in 1 ml of 0.5M 2-(N-morpholino)ethanesulfonic acid (MES) buffer (pH=6.3) and incubated at 50° C. for 10 min. The mixture was added to 4 ml of amino-functionalized BNF susceptors (FIG. 2 II) (34.4 mg Fe, 48 mg particles) and shaken on a Labquake® mixer at room temperature for 2 h. The resulting BNF-PEG-COOH susceptors were washed three times with deionized water by magnetic separation and filtered through a 0.22 mm Millex-GP filter to give a 5 ml suspension (FIG. 2 III) with an iron concentration of 5.3 mg/ml.

Five hundred microliters of this suspension were mixed with a solution of 0.6 mg (3 μmol) EDC and 1.2 mg (10 μmol) N-hydroxysuccinimide (NHS) in 125 ml 0.5M MES buffer (pH=6.3) and shaken for 90 min at room temperature. After washing the particles twice with phosphate buffered saline (PBS) (pH=7.4) with magnetic separation, 100 ml of rabbit anti-goat IgG (400 mg/ml) was added. The mixture was shaken for 3 h, and the reaction was quenched by the addition of glycine in PBS and washed three times with PBS buffer by magnetic separation to give 1.2 ml of antibody conjugated BNF-PEG-COOH susceptors (FIG. 2 IV) with an iron concentration of 2 mg/ml.

### Example 4

### Synthesis of BNF Susceptors with ANB Groups on the Surface and Conjugation with Rabbit Anti-Goat IgG

All reactions involving N-5-azido-2-nitrobenzoyloxysuccinimide (ANB-NOS) were carried out under protection from light. ANB-NOS (1.5 ml of 1 mM) in carbonate solution (pH=8.5) was mixed with 500 μl of amino-functionalized BNF susceptors (FIG. 2 II) (4.3 mg Fe, 6 mg particles) and shaken at room temperature for 2 h. The BNF-ANB susceptors were washed twice with phosphate buffered saline (PBS) (pH=7.4) with magnetic separation giving a 1 ml suspension (FIG. 2 V) with an iron concentration of 3.8 mg/ml. One hundred microliters of rabbit anti-goat IgG (400 mg/ml) were added to the susceptors, and the mixture was shaken for 2 h while exposing to 302 nm light. The reaction was quenched with the addition of a solution of glycine in PBS and was then washed three times with PBS buffer with magnetic separation to give a 1.2 ml suspension of antibody conjugated BNF-ANB susceptors (FIG. 2 IV) with an iron concentration of 2.1 mg/ml.

### Example 5

### Sulfhydryl Labeling of Rabbit Anti-Goat IgG

Rabbit anti-goat IgG was labeled with sulfhydryl groups by reaction with N-succinimidyl-5-acetylthioacetate (SATA) followed by deacetylation with hydroxylamine using the manufacturer's instructions. Five hundred microliters of rabbit anti-goat IgG (400 mg/ml) and 2 ml of a SATA solution in dimethylformamide (DMF) (4 mg/ml) were incubated at room temperature for 30 min. Twenty microliters of a hydroxylamine solution in 0.1M PBS buffer, 0.005M EDTA (5 mg/100 ml) were added to the antibody solution and incubated at room temperature for 2 h. The antibody was then purified with a G25 column. The concentration of the SH-labeled rabbit anti-goat IgG was determined by absorption measurement at 280 nm.

### Example 6

### Synthesis of BNF Susceptors with 2-Pyridyldisulfide Groups on the Surface and Conjugation with Sulfhydryl-Labeled Rabbit Anti-Goat IgG

Five hundred microliters of amino-functionalized BNF susceptors (FIG. 2 II) (4.3 mg Fe, 6 mg particles) were mixed with 500 ml of PBS buffer and 800 μl of 20 mM N-succinimidyl 3-(2-pyridyldithio)propionate (SPDP). The mixture was shaken for 1 h at room temperature and washed three times with phosphate buffered saline (PBS) by magnetic separation to yield a 1 ml suspension of BNF-SPD susceptors (FIG. 2 VII) with an iron concentration of 4.1 mg/ml. The number of attached 2-pyridyldisulfide groups was measured using the manufacturer's instructions. One hundred microliters of BNF-SPDP susceptors (FIG. 2 VII) (4.1 mg/ml Fe) in PBS and 100 ml of reference amino-functionalized BNF susceptors (FIG. 2 II) (4.0 mg/ml Fe) in PBS each were incubated with 100 ml of 50 mM dithiothreitol (DTT) solution in PBS and shaken at room temperature for 15 min. The susceptors were then centrifuged at 15,000 rpm for 15 min and the absorption of the supernatants was measured at 343 nm and recorded. The difference of the absorption of the supernatants of BNF-SPDP susceptors and reference amino-functionalized BNF susceptors was used to calculate the 2-pyridyldisulfide concentration using a molar extinction coefficient of 8080 M⁻¹ cm-¹ at 343 nm. The resulting concentration of 2-pyridyldisulfide groups on the surface of the BNF-SPDP susceptors (FIG. 2 VII) was 37 nmol/mg iron.

Nine hundred microliters of BNF-SPDP susceptor (FIG. 2 VII) suspension with an iron concentration of 4.1 mg/ml was incubated with 1 ml of sulfhydryl-modified rabbit anti-goat IgG (96 μg/ml) in PBS for 3 h at room temperature and was washed three times with PBS buffer by magnetic separation resulting in 1 ml suspension of antibody conjugated BNF-SPDP susceptors (FIG. 2 VIII) with an iron concentration of 3.6 mg/ml.

### Example 7

### Synthesis of BNF Susceptors with Maleimide Groups on the Surface and Conjugation with Sulfhydryl-Labeled Rabbit Anti-Goat IgG

7.5 mmol N-ethyl-N′-(3-dimethylaminopropyl)carbodiimide hydrochloride (EDC) and 7.7 mmol N-maleoyl-b-alanin were dissolved in 125 ml 0.5M 2-(N-morpholino)ethanesulfonic acid (MES) buffer (pH=6.3) and incubated at 50° C. for 10 min. The mixture was added to 500 ml of amino-functionalized BNF susceptors (FIG. 2 II) (4.3 mg Fe, 6 mg particles) and shaken at room temperature for 2 h. The resulting BNF-maleimide susceptors (FIG. 2 IX) were washed three times with deionized water by magnetic separation and filtered through a 0.22 mm Millex-GP filter to give a 1 ml suspension with an iron concentration of 4.0 mg/ml. This BNF-maleimide susceptor suspension was incubated with 1 ml of sulfhydryl-modified rabbit anti-goat IgG (96 mg/ml) in phosphate buffered saline (PBS) for 3 h at room temperature, and washed three times with PBS buffer by magnetic separation resulting in 1 ml suspension of antibody conjugated BNF-maleimide susceptors (FIG. 2 X) with an iron concentration of 3.8 mg/ml.

### Example 8

### Immunoreactivity Study of the BNF-Antibody Susceptor Conjugates

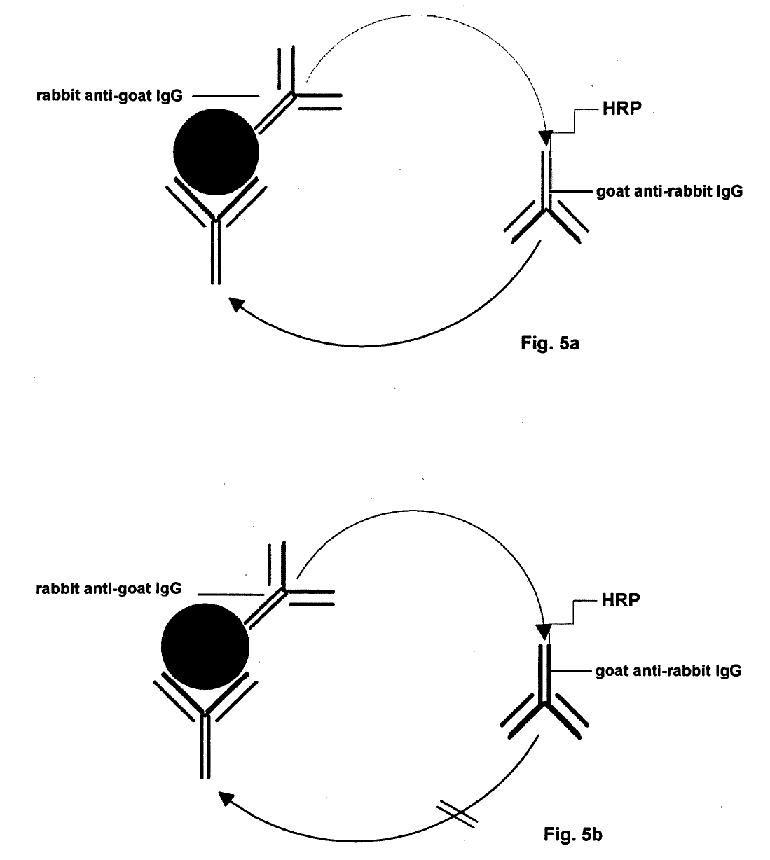

A two-step immunoassay was developed to compare the efficacy of the different antibody binding strategies depicted in FIG. 2. In the first step, the total amount of bound rabbit anti-goat IgG was characterized using a HRP-labeled goat anti-rabbit IgG as a secondary antibody. This secondary antibody can interact with the rabbit antigoat-particle conjugates in various ways. FIG. 5a is a schematic illustrating two ways in which the secondary goat anti-rabbit antibody may interact with the rabbit anti-goat antibody conjugated to the surface of the BNF susceptor. In the second step, the total amount of bound immunoreactive rabbit anti-goat IgG was determined using HRP-labeled goat anti-mouse IgG. This secondary antibody can only be bound on the susceptor surface if the primary antibody is attached in the correct orientation. FIG. 5b is a schematic illustrating that a goat anti-mouse antibody can only interact with a rabbit anti-goat antibody conjugate when the rabbit anti-goat is in the proper orientation.

The rabbit anti-goat IgG labeled susceptors (FIGS. 2 IV, VI, VII and X) were washed twice with 0.01M phosphate buffered saline (PBS) (pH=7.4) containing 0.05% polysorbate 20 (Tween 20). The susceptors were blocked with 1 ml of 0.0M PBS (pH=7.4) containing 0.05% polysorbate 20 and 1% bis(trimethylsilyl)acetamide (BSA) for 2 h shaking at room temperature. After washing three times with 0.01M PBS (pH=7.4) containing 0.05% polysorbate 20, the iron concentration of the susceptor suspensions was characterized and adjusted so that each suspension had the same iron concentration. After incubation with HRP-labeled goat anti-rabbit IgG for 2 h at room temperature on a rocker the susceptors were centrifuged at 23,000 rpm for 15 min. A 200 ml aliquot of the supernatant that contained the unbound HRP-labeled goat anti-rabbit IgG was developed with 50 ml 2.2 mM 1,2-phenylenediamine dihydrochloride (OPD) containing 0.012% 30% hydrogen peroxide at room temperature for 10 min. The reaction was stopped by adding 50 ml of 1.8M sulfuric acid and the absorption was measured at 492 nm. The amount of rabbit anti-goat IgG that was covalently bound to the surface of the susceptors was determined by monitoring the disappearance of the secondary HRP-labeled goat anti-rabbit IgG after incubation with the rabbit anti-goat IgG labeled susceptors (FIGS. 2 IV, VI, VIII and X).

The immunoreactive antibody fraction on the susceptor surface was determined using the HRP-labeled goat anti-mouse IgG as a secondary antibody in the same manner as described above. The amount of bound HRP-labeled goat anti-mouse IgG secondary antibody represents only the immunoreactive primary antibody molecules on the susceptor surface. The amount of non-specifically bound HRP-labeled goat anti-rabbit and goat anti-mouse antibodies was negligible when compared against the control amino-functionalized BNF susceptors (FIG. 2 II).

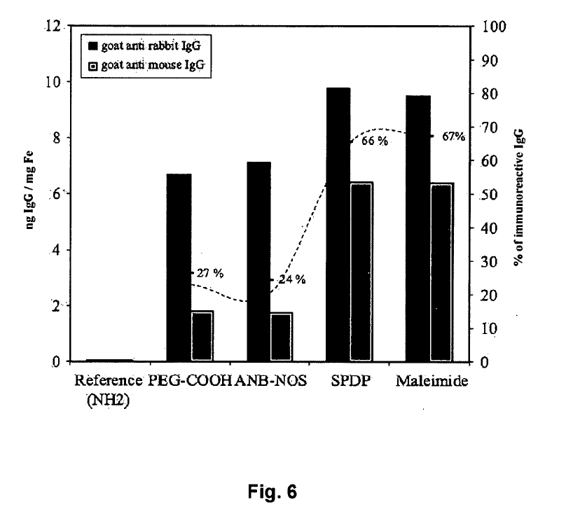

A comparison of the immunoassay results of the four different antibody-susceptor conjugation strategies to obtain the antibody-labeled BNF susceptors (FIGS. 2 IV, VI, VIII and X) is shown in FIG. 6. FIG. 6 is a bar graph depicting the total bound antibody compared to the immunoreactivity per mg of iron with antibody conjugated susceptors prepared using the strategies illustrated in FIG. 2. The total amount of antibody bound to the susceptor surface using SPDP and maleimide-based conjugation methods is 28-30% higher than for antibodies conjugated using PEG-COOH or ANB groups on the surface. While the PEG-COOH and ANB-NOS based conjugation strategies only result in about 24-27% of immunoreactive antibody on the susceptor surface, the SPDP and maleimide-based methods lead to a percentage of 66-67% of immunoreactive antibody related to the total amount of covalently attached primary antibody.

The experimental results demonstrate that the synthesis of stable high SAR magnetic susceptors is possible with biocompatible materials. Further, it is possible to conjugate antibodies to the susceptors using a variety of techniques, while maintaining particle integrity and colloidal stability.

### Example 10

### Preparation and Characterization of BNF-Antibody Susceptor Conjugates

Trastuzumab is first converted to thiols using 2-iminothiolane. Then, a total of 1 to 2 ml of about 2×10⁻⁵ M thiolated trastuzumab in degassed phosphate buffered saline (PBS) is added to 20 ml (400 mg) of BNF-maleimide susceptors in degassed PBS. The reaction mixture is shaken for at least one hour at room temperature. Sufficient N-ethylmaleimide (NEM) is then added to achieve a 10 mM solution in that reagent. The mixture is shaken for about 40 minutes and then subjected to magnetic separation for about 30 minutes. The supernatant is removed and fresh buffer added for the next magnetic separation. Washing is repeated two or more times and the susceptor conjugates are then resuspended in 2 mM mercaptoethanol. The suspension is then shaken and washing sequence continued for two or more washes at which point the antibody-functionalized susceptor conjugates are resuspended in PBS.

Iron analysis was performed on the final suspension of susceptor conjugates. Comparison of the measured iron concentration to that of a 20 mg/ml particle standard (from the same Micromod lot) facilitated final volume adjustment to obtain approximately 20 mg/ml of the susceptor conjugate.

Specific Absorption Rate (W/g of iron) (SAR) is obtained using FISO Technologies UMI4 Universal Multichannel Instrument with FISO Commander 2 Standard Edition, calibration curve by Techtronix TDS 2024B.

Binding capacity of the susceptor conjugates was determined using a direct cell binding assay adapted from a method published by Lindmo et al. Immortalized human tumor cells expressing target antigens were harvested from tissue cultures. Cells were washed and resuspended in blocking buffer at a cell concentration sufficient to provide antigen excess in the reaction tube, typically from 20 to 50 million per tube, depending on antigen expression on the cell. Multiple microtubes were prepared with increasing cell concentrations. A small quantity of susceptor-antibody conjugate, typically less than 2 μg, was added to each microtube. Cells and susceptor-antibody conjugates were incubated for 4 hours at 4° C. with constant mixing. Unbound susceptor conjugates were separated from bound using 5 micron nylon filters. The iron content of the bound portion was calculated by measuring iron in the unbound portion and subtracting from the initial quantity of iron added to the test. Results were reported as the portion of bound susceptor conjugates plotted against cell concentration at infinite antigen excess. Nonspecific binding of susceptor conjugates was determined by performing the same assay using susceptor conjugates bound to an irrelevant antibody.

In preparation for binding studies, adherent cells (HT-29 for ING-1, SKBR-3 for herceptin) were grown to confluency in 96-well plates in appropriate medium.

For saturation binding experiments, 125-I labeled ligands (ING-1, herceptin or BNF-susceptor conjugates) in concentrations ranging from 0-1000 nM were prepared by serial dilutions (typically 1:1 or 1:3) across rows of a 96-well plate, a typical final volume per well being 100 ul. Plates were incubated for 2-20 hours at 4° C. or room temperature (with or without shaking) to achieve equilibrium binding. After binding, the wells were washed 3 times with 100 ul wash buffer (typically 1×PBS/0.1% BSA or McCoy's 5a/10% FCS). Bound counts were stripped from the wells with 100 ul 0.1N NaOH for 20-30 minutes at room temperature. Stripping solution was transferred to tubes and counted in a gamma counter. Counts were entered into GraphPad Prism, which performed non-linear curve fitting and computed Bmax and Kd values.

For competitive binding experiments, 125-I labeled high-specific activity ligand (as trace-typical [trace]=0.1-5.0 nM) was mixed with unlabeled ligand in unlabeled ligand concentrations ranging from 0-1000 nM prepared by serial dilutions across rows of a 96-well plate, a typical final volume per well being 100 ul. Plates were incubated for 2-20 hours at 4° C. or room temperature (with or without shaking) to achieve equilibrium binding. After binding, the wells were washed 3 times with 100 ul wash buffer (typically 1×PBS/0.1% BSA or McCoy's 5a/10% FCS). Bound counts were stripped from the wells with 100 ul 0.1N NaOH for 20-30 minutes at room temperature. Stripping solution was transferred to tubes and counted in a gamma counter. Counts were entered into GraphPad Prism, which performed non-linear curve fitting and computed EC50 and Ki values.

TABLE 1
Summary of ING-1-BNF, HER-BNF, and Control (IgG) Conjugate Preparations.

| | | | BNF-ING² | BNF-ING² |
|---|---|---|---|---|
| | BNF-Her | BNF-IgG¹ | Lot#1 | Lot#2 |
| Ab per cell | 20 | 30 | 30 | 24 |
| Z-Average/PDI | 158/0.170 | 155/0.133 | 158/0.177 | 156/0.206 |
| Fe mg/ml | 11.8 | 12.9 | 13.7 | 13 |
| Solid mg/ml | 18.6 | 19.7 | 21.7 | 19.5 |
| SAR 1:1 with | 249 w/g Fe | 247 w/g Fe | 235 w/g Fe | 274 w/g Fe |
| water | | | | |
| SAR undiluted | 133 w/g Fe | | 201 w/g Fe | 278 w/g Fe |
| Immunoreactivity | SKBR-3 Cell | SKBR-3 cell | HT-29 cell | HT-29 cell |
| | bound: 64-68% | background: | bound 80% | bound 75% |
| | background: | 21-36% | background | background |
| | 21-36% | | 26% | 25% |
| Competitive | delta EC50: | no binding | Not | Not |
| binding I-125 | 1.0 to 1.1 | | Determined | Determined |
| log EC50 BNF-Ab | | | | |
| vs naked Ab | | | | |

¹Non-binding control susceptor conjugate prepared using the using commercially-available polyclonal human IgG, according to the procedure described above.
²Susceptor conjugate prepared using c humanized anti-EpCAM antibody (designated ING-1), according to the procedure described above.

Although the invention has been described in considerable detail with reference to certain preferred aspects thereof, other versions are possible. Therefore the spirit and scope of the appended claims should not be limited to the description and the preferred versions contained within this specification.

What is claimed is:

1. A ligand conjugated particle comprising: an amino-functionalized nanoparticle forming a single magnetic domain; at least one linker in communication with the amino-functionalized nanoparticle; and at least one ligand coupled to the amino-functionalized nanoparticle or the linker.

2. The ligand conjugated particle of claim 1, wherein the magnetic nanoparticle comprises bionized nanoferrite.

3. The ligand conjugated particle of claim 1, wherein the magnetic nanoparticle has an iron content of greater than 50% (w/w).

4. The ligand conjugated particle of claim 1, wherein the linker is a bifunctional compound.

5. The ligand conjugated particle of claim 1, wherein the linker is a multi-subunit composition comprising one or more subunit selected from a haloalkyl, epoxide, vinyl heterocumulene, epoxypropene, polyethylene glycol, polypropylene or combination thereof.

6. The ligand conjugated particle of claim 1, wherein the linker comprises one or more hydrophilic subunit.

7. The ligand conjugated particle of claim 1, wherein the linker comprises a mixture of chemically different compounds.

8. The ligand conjugated particle of claim 1, wherein the linker comprises at least one diepoxide, at least one poly(ethylene glycol) epoxyether, at least one poly(ethylene glycol) diglycidyl ether, at least one epichlorohydrin or combination thereof.

9. The ligand conjugated particle of claim 1, wherein the linker comprises a mixture of epichlorohydrin and poly(ethyleneglycol)diglycidyl ether.

10. The ligand conjugated particle of claim 1, wherein the amino-functionalized particle comprises substructures, said substructures comprising at least one linker, at least one ligand, at least one chelator or a combination thereof.

11. The ligand conjugated particle of claim 1, wherein the linker comprises one or more terminal reactive group selected from amine, thiol, hydrazine, azide, disulphide, sulphonic acid, carboxylic acid, maleimide or combination thereof.

12. The ligand conjugated particle of claim 11, wherein the reactivity of terminal groups is based on substitution or addition chemistry.

13. The ligand conjugated particle of claim 11, wherein the carboxylic acid is poly(ethylene glycol)ether based carboxylic acid.

14. The ligand conjugated particle of claim 11, wherein the azide is 5-azido-2 nitrobenzamide.

15. The ligand conjugated particle of claim 11, wherein the disulphide is 3-(2-pyridyldithio)propionamide.

16. The ligand conjugated particle of claim 11, wherein the maleimide is 1,2-diacylethene or 3-maleimidylpropionamide.

17. The ligand conjugated particle of claim 1, wherein the ligand is an antibody.

18. The ligand conjugated particle of claim 1, wherein the ligand is modified by incorporation of a group selected from a thiol or an amine.

19. The ligand conjugated particle of claim 1, wherein the ligand is modified with N-succinimidyl-S-acetylthioacetate.

20. The ligand conjugated particle of claim 1, further comprising a biocompatible coating.

21. The ligand conjugated particle of claim 20, wherein the surface of the amino-functionalized nanoparticle forms the biocompatible coating.

22. The ligand conjugated particle of claim 1, wherein the particle is a thermotherapeutic agent.

23. A ligand conjugated particle comprising: a functionalized magnetic nanoparticle and at least one linker in communication with the functionalized magnetic nanoparticle wherein the specific absorption rate (SAR) of said ligand conjugated nanoparticle is at least 5 fold higher than 20 nm Nanomag®-D-spio particles.

24. The ligand conjugated particle of claim 23, further comprising a ligand coupled to the functionalized magnetic nanoparticle or the linker.

25. A method of treating disease in a subject, comprising administering to the subject an effective amount of the ligand conjugated particle of claim 1.

26. A method for preparing a ligand conjugated particle comprising: (i) functionalizing a particle forming a single magnetic domain with amino or nitro groups; (ii) contacting the functionalized particle with a linker; and (iii) coupling a ligand to the particle or the linker to form a ligand conjugated particle.

27. The method for preparing a ligand conjugated particle of claim 26, wherein the nanoparticle forming a single magnetic domain comprises bionized nanoferrite.

28. The method for preparing a ligand conjugated particle of claim 26, wherein the nanoparticle forming a single magnetic domain has an iron content of greater than 50% (w/w).

29. The method for preparing a ligand conjugated particle of claim 26, wherein the linker is a bifunctional compound.

30. The method for preparing a ligand conjugated particle of claim 26, wherein the linker is a multi-subunit composition comprising one or more subunit selected from a haloalkyl, epoxide, vinyl heterocumulene, epoxypropene, polyethylene glycol, polypropylene or combination thereof.

31. The method for preparing a ligand conjugated particle of claim 26, wherein the linker comprises one or more hydrophilic subunit.

32. The method for preparing a ligand conjugated particle of claim 26, wherein the linker comprises a mixture of chemically different compounds.

33. The method for preparing a ligand conjugated particle of claim 26, wherein the linker comprises at least one diepoxide, at least one poly(ethylene glycol)epoxyether, at least one poly(ethylene glycol)diglycidyl ether, at least one epichlorohydrin or combination thereof.

34. The method for preparing a ligand conjugated particle of claim 26, wherein the linker comprises a mixture of epichlorohydrin and poly(ethyleneglycol)diglycidyl ether.

35. The method for preparing a ligand conjugated particle of claim 26, wherein the linker comprises one or more terminal reactive group selected from amine, thiol, hydrazine, azide, disulphide, sulphonic acid, carboxylic acid, maleimide or combination thereof.

36. The method for preparing a ligand conjugated particle of claim 35, wherein the reactivity of terminal groups is based on substitution or addition chemistry.

37. The method for preparing a ligand conjugated particle of claim 35, wherein the carboxylic acid is poly(ethylene glycol)ether based carboxylic acid.

38. The method for preparing a ligand conjugated particle of claim 35, wherein the azide is 5-azido-2 nitrobenzamide.

39. The method for preparing a ligand conjugated particle of claim 35, wherein the disulphide is 3-(2-pyridyldithio)propionamide.

40. The method for preparing a ligand conjugated particle of claim 35, wherein the maleimide is 1,2-diacylethene or 3-maleimidylpropionamide.

41. The method for preparing a ligand conjugated particle of claim 26, wherein the ligand is an antibody.

42. The method for preparing a ligand conjugated particle of claim 26, wherein the ligand is modified by incorporation of a group selected from a thiol or an amine.

43. The method for preparing a ligand conjugated particle of claim 26, wherein the ligand is modified with N-succinimidyl-S-acetylthioacetate.

44. The method for preparing a ligand conjugated particle of claim 26, wherein the functionalization step occurs at a pH of between about 7 and about 9.

45. The method for preparing a ligand conjugated particle of claim 26, further comprising the additional step of washing the ligand conjugated particle with an aqueous buffer solution.

46. The method for preparing a ligand conjugated particle of claim 26, further comprising the additional step of sterilizing the ligand conjugated particle.

47. The method for preparing a ligand conjugated particle of claim 26, wherein the washing step occurs at a pH of between about 5 and about 8.

48. The method for preparing a ligand conjugated particle of claim 26, wherein the step of coupling the ligand to the particle or the linker to form the ligand conjugated particle occurs within 12 hours of the step of contacting the functionalized particle with the linker.

49. The method for preparing a ligand conjugated particle of claim 26, wherein the ligand conjugated particle ranges in size from 10-80 nm.

50. A nanoparticle for thermotherapy prepared by a process comprising the steps: (i) functionalizing a particle forming a single magnetic domain with amino or nitro groups; (ii) contacting the functionalized particle with a linker; and (iii) coupling a ligand to the particle or the linker to form a ligand conjugated particle.
